(19) United States
(12) Patent Application Publication
(10) Pub. No.: US 2025/0352855 A1
(43) Pub. Date: Nov. 20, 2025

(54) Apparatus, System, and Method of Testing and Improving Balance

(71) Applicant: Balanse, Inc., Olympia, WA (US)

(72) Inventor: William F. Dirkes, Olympia, WA (US)

(73) Assignee: Balanse, Inc., Olympia, WA (US)

(21) Appl. No.: 19/212,642

(22) Filed: May 19, 2025

Related U.S. Application Data
(60) Provisional application No. 63/649,911, filed on May 20, 2024.

(51) Int. Cl. A63B 24/00

(52) U.S. Cl. CPC A63B 24/0062; A63B 2220/803; A63B 2220/836

(57) ABSTRACT

A method, system, and/or apparatus for measuring user balance with sensors of a mobile device such as a mobile phone or wearable while showing a video or animation coaching various balance exercises. A filter can be utilized to merge accelerometer and gyroscope data to reduce noise. A quaternion-based orientation pipeline can compute the device's pitch, roll, and overall orientation. The system can calculate stability scores by measuring factors such as variance or root-mean-square of pitch and roll fluctuations, acceleration magnitudes, and/or jerk/fall events. If the user experiences sudden/large movements, the system penalizes the user's score to discourage risky behavior. The user may be asked to stand in a baseline posture to measure typical involuntary arm tremor, truncal instability, or normal minimal device motion, which is used to normalize subsequent balance measurements. The system gamifies the experience by providing real-time feedback and awarding points, achievements, and an overall score.

### CROSS-REFERENCE TO RELATED APPLICATION

This application claims priority from and benefit of the filing date of U.S. provisional application Ser. No. 63/649,911 filed May 20, 2024, and the entire disclosure of said provisional application is hereby expressly incorporated by reference into the present specification.

### BACKGROUND

Balance is important yet often underappreciated, naturally degrading as a person ages. A person over 50 years of age who cannot stand on one leg for ten seconds is at four-fold increased risk of death over the next 7 years compared to a similarly aged person who can complete this brief test. Poor balance is associated not only with increased mortality, but also increased rate of depression and lower psychological, physical, and social quality of life. According to the Center for Disease Control (CDC), in adults over 65 years old, nearly one in four will fall each year, resulting in nearly 39,000 deaths in 2021, which represents the leading cause of injury and injury death. Although balance worsens with age secondary to worsened proprioception and neural connectivity, research has demonstrated that there are methods to improve balance.

Physical therapy and rehabilitation programs focus on improving balance but are only initiated once balance, mobility, and strength are significantly diminished, usually after severe illness or injury has already occurred. Prevention is critical, and although doctors have methods of screening patients and initiating preventative measures in high-risk patients, an accessible and appealing platform focused on improving balance before severe impairment is advantageous.

There is a need for a more accessible platform, such as a mobile or wearable device, that can objectively measure, track, and potentially improve a user's balance by providing real-time feedback. Recent advances in sensor fusion algorithms, including quaternion-based orientation filtering and Kalman filtering, can be used to generate more robust and drift-resistant estimates of device orientation (pitch and roll) and movement. By incorporating these algorithms and an engaging, gamified user experience, a broader audience can be reached, including users ranging from older adults who need safe exercises to athletes seeking advanced balance training.

### SUMMARY

In one embodiment, this disclosure relates to a method, system, and apparatus of objectively measuring user balance with the sensors built-in to a mobile device such as a mobile phone and/or a wearable device such as a smart device (e.g., smart watch or similar wearable) while showing a user engaging video or animation coaching of various balance exercises. A Kalman filter can be used to merge accelerometer and gyroscope data to reduce noise, while a quaternion-based orientation pipeline can compute the device's pitch, roll, and overall orientation more accurately.

The system calculates stability scores by measuring factors such as variance or root-mean-square of pitch and roll fluctuations, acceleration magnitudes, and/or jerk/fall events. If the user experiences sudden or large movements, the system penalizes the user's score to discourage risky behavior. The user may also be asked to stand in a baseline posture for a brief period to measure typical involuntary arm tremor, truncal instability, or normal minimal device motion, which is then used to normalize subsequent balance measurements. The system gamifies the experience by providing real-time feedback and awarding points, achievements, and an overall score.

The application covers both real-life instructor videos and simpler, possibly cartoon-style or symbolic animations demonstrating the exercises on screen. Exercises can be static (e.g., stand on one leg) or dynamic (e.g., squat or lunge), and the algorithm can ignore or diminish certain axes of motion if they represent intentional movement rather than instability. Additionally, the system can measure more advanced metrics such as repeated forward-back reaching distances and the smoothness of the movement.

Finally, the application describes a user interface with multiple modes (e.g., easy, moderate, advanced) tied to user-chosen surfaces: chair assistance, floor, balance pad, disc, wobble board, etc. By combining measured sensor data and a library of known exercises, the system computes an overall “balance score” or “stability score,” helps track the user's progress, and recommends safer alternatives to those with low stability scores to reduce risk of falls.

### BRIEF DESCRIPTION OF THE DRAWINGS

FIG. 1 shows a representative or sample screenshot of the app (such as a home screen) that can be displayed on a mobile phone or other mobile device, prompting the user, for example, to initiate the app.

FIG. 2 is a subsequent screenshot of different selectable exercises, and/or prompts for selecting a desired level of difficulty, starting the selected exercise, etc.

FIG. 3 is a screenshot depicting an instructor performing a selected exercise and illustrating one preferred manner of using the smart/mobile device by the instructor during the selected exercise (in this example the user should be holding the device D by hand with an outstretched arm while viewing the display screen of the device D and mimicking the exercise or pose displayed on the device). The device D takes accelerometer and/or gyroscope data and displays a measure of stability on the screen, providing real-time feedback to the user. The demonstration of the exercise for the user may take the form of a video as depicted here, an animation, or a picture.

FIG. 3A is a drawing of a screenshot that is similar to FIG. 3 but shows an alternative embodiment.

FIG. 4 is a representative image or screenshot illustrating, for example, weekly score trends, score breakdowns, summaries of individual exercises, etc.,

FIG. 5 provides a collection of information/data relating to the activities and progress of the user.

FIG. 6 illustrates representative selection items that allow the user to customize a routine.

FIG. 7 shows a representative screenshot of various settings, biographical information, ability to refer a friend, leave a review, or contact the app provider or administrator.

FIG. 8 illustrates an example of a suitable device D for implementing an apparatus, system, and method of testing and improving balance in accordance with one embodiment of the present development.

### DETAILED DESCRIPTION

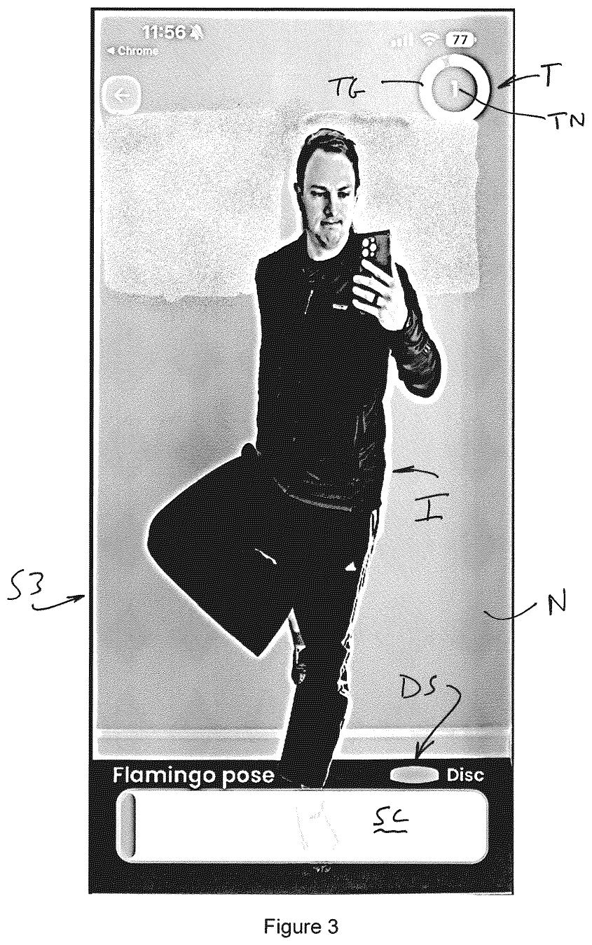

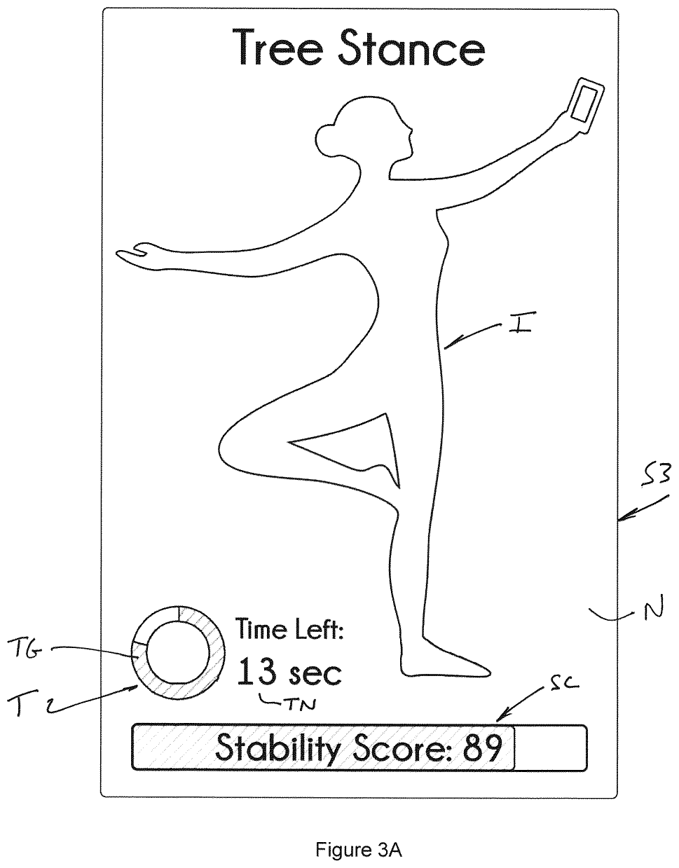

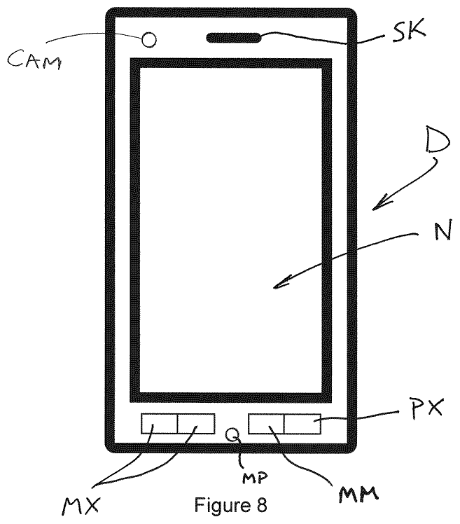

One version of the present disclosure is a software application which runs on a device D (see FIG. 8) which contains a visual output display screen N and associated device movement sensors MX such as an accelerometer, gyroscope, and/or the like, or the software application can run on multiple devices which together contain a display screen and accelerometer, gyroscope, and/or other movement sensors. The device(s) D may comprise a mobile device such as a mobile phone such as an Apple iPhone or an Android mobile phone in one non-limiting embodiment but may also be a device such as an iPod touch, tablet, or a combination of a phone, smart watch, Fitbit, or related component(s) in which and one or more of the devices could serve as the sensor and screen. The user holds or otherwise maintains the device D preferably one to two feet from their body with a fully or partially outstretched arm, or as far as an arm's length away, fashioning or arranging the output display screen N of the device D to be viewable to the user during the exercise as shown or demonstrated by the instructor I in FIG. 3. The user is typically prompted to perform various balance or mobility exercises or poses via videos and/or animations and/or photographs/images displayed on the screen N while holding the device D with a fully or partially outstretched arm as described. As used herein, the term “video” is intended to encompass captured real-world moving images and also computer-generated or other animations that depict or represent a moving person or other object, while the term “image” is intended to encompass a photograph of a real-world person or other object, or a drawing or computer-generated image or representation of a person or other object. An algorithm comprising a series of computer executable instructions is stored within a memory MM (computer readable media) contained within the mobile device D and is implemented or carried out within an electronic processor PX of the device D and/or in an external computer server. The processor PX receives as input the movement measurements from the device sensors MX (e.g., X, Y, and Z axis movement and/or acceleration data). The processor PX carries out the stored instructions to perform the method and steps described herein and can output a “stability score” and/or other stability data to the user visually on the device screen N and/or using audible data output by an audio output speaker SK of the device D. The stability score can be calculated by the processor PX and output by the device D after the user performs one or a plurality of balance exercises/poses as described above, and/or the stability score and can calculated and/or output by the device D in real-time to the user while the user is performing the one or more balance exercises/poses.

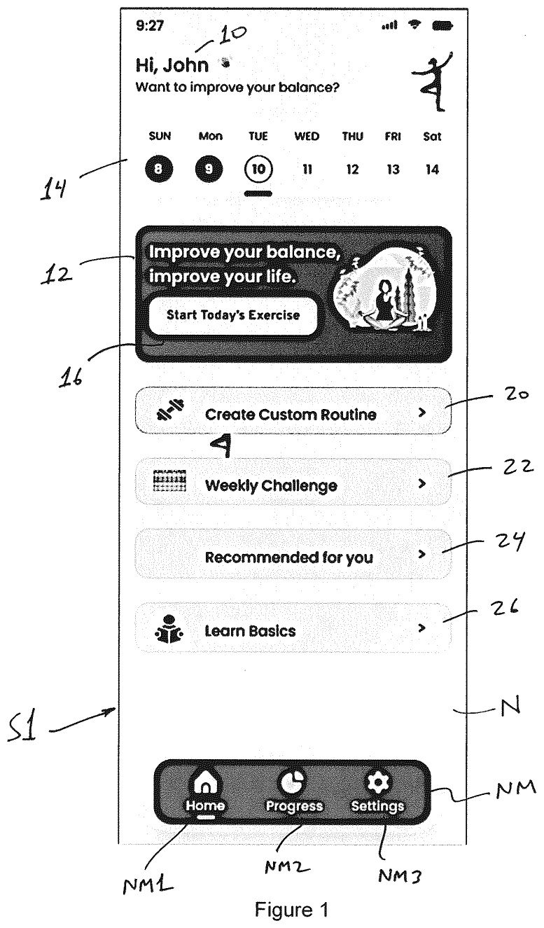

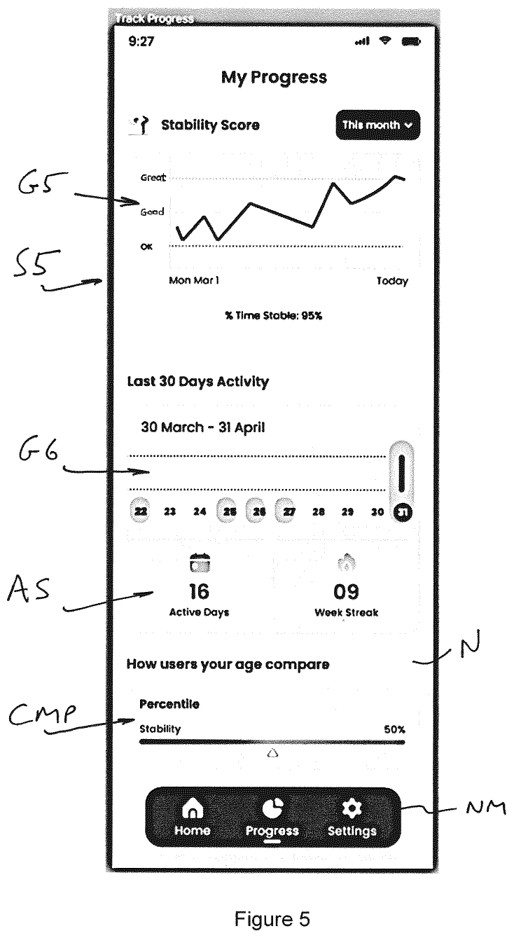

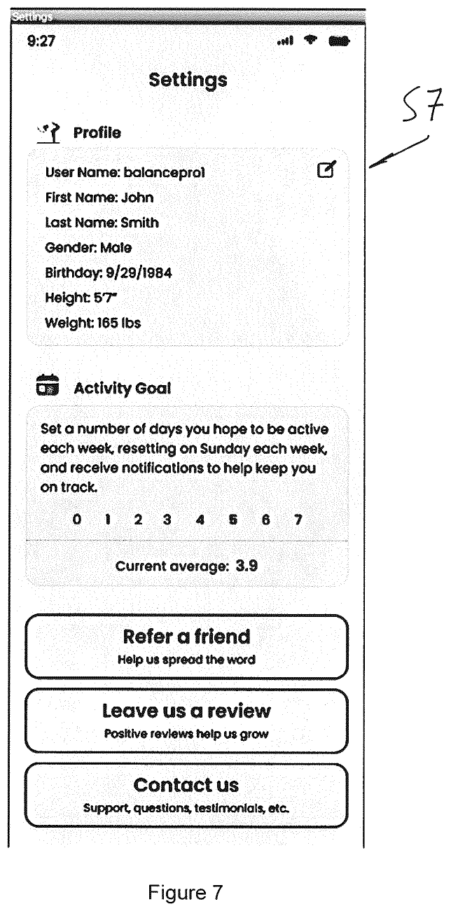

As shown in FIG. 1, the display or screen N of the mobile device D can display a home screen S1 that provides general information to a user such as the user's name 10, motivational message 12, a schedule of past or upcoming exercises to be performed 14, and/or a Start Today's Exercises icon 16 to be selected by the user (tapped or otherwise selected or activated on the device screen N by the user) to begin balance training for the user for today. The Start Today's Exercises icon 16 and other user selectable icons or buttons can be tapped or otherwise selective by the user on the screen of the device D and/or the device D can be configured to accept verbal commands such as “Start” or the like spoken by the user into a microphone MP of the device D. One or more additional user selectable icons can be displayed such as a create custom routine icon 20 which when selected allows the user to create a custom series of one or more exercises to be performed at one or more selected days and times, a weekly challenge icon 22 that when selected allows the user to compare his/her progress against certain challenge goals, a recommended for you icon 24 that when selected suggest certain exercises and/or poses to be performed by the user based upon the user's balance proficiency level and/or usage history as tracked by the device D, and/or a learn basics training icon 26 that will provide demonstrations and/or educational content to the user on the mobile device D. The home screen S1 (and/or any other screen displayed by the device D) can also include a navigation menu NM that that allows the user to select home, progress, or settings icons NM1,NM2,NM3 to immediately return the display to the home screen S1, the progress screen S5 of FIG. 5, or the settings screen S7 of FIG. 7, respectively.

In use, the user holds or otherwise supports and maintains the device D preferably one to two feet from the trunk of their body, or as far as arm's length away, fashioning the screen to be viewable by the user as demonstrated on the screen N of the user's device D by the instructor I in FIG. 3 using a still image and/or a video image, with our without sound that can provide additional instructions and/or guidance to the user. The user is prompted visually on the display screen N of the device D and/or via audible messages or tones output by the speaker(s) SK of the mobile device D to perform one or more various balance and/or mobility exercises and/or poses via videos and/or images displayed on the device screen N that the user will mimic to the best of the user's ability while seeking to hold the device D as steady as possible for the duration of time shown and/or indicated by a timer T which can count or move up or down using numbers TN and/or other visual indicia such a graph TG or the like. Timer T can also comprise audible time information output by the device speaker SK to indicate to the user the amount of time remaining in the pose/exercise being performed by the user.

The device D and method can comprise either or both: (i) a real-time algorithm which provides users with immediate feedback on user stability; and/or (ii) a post-exercise algorithm which provides users with feedback on user stability after the user has completed one or more exercises/poses. The real-time and/or post-exercise algorithms can be implemented on the device D or an associated device or server which receives as input movement measurements from the accelerometer and/or gyroscope sensors MX of the device D while the user while is performing each balance/mobility exercise or pose. In both cases, pre-processing of the data to account for signal noise is advantageous, such as with a fourth order Butterworth filter.

The real-time algorithm can provide users with immediate stability performance metrics on screen N and is depicted in one embodiment as SC in FIG. 3. The recent velocity data from the accelerometer and rotation data from the gyroscope may be smoothed using an exponential moving average. The movements may also have scalar multiplication performed for each accelerometer x, y, z or gyroscope gx, gy, gz dimension as described below to augment, diminish, or ignore certain directions of movement and then be normalized for a specific range. In the illustrated example of FIG. 3, the stability performance metric SC is shown as a bar graph but, as shown in the alternative embodiment of FIG. 3A, it can additionally or alternatively include an alphanumeric element such as “A,” “B,” “C,” etc. or “10,” “9,” “8,” etc. or a combination of letters and numbers including an adjective description such as “GOOD,” “FAIR,” “POOR,” etc. Additionally, a fusion of the accelerometer and gyroscope data using a Kalman filter can output pitch forward/back or roll left/right of the device.

The post-exercise algorithm can calculate multiple measures of stability performance and then uses these metrics to devise an overall “stability score” while also subtracting a user's pre-measurement of arm sway and tremors. A higher stability score indicates better performance at holding mobile device D was stable by the user while performing each balance/mobility exercise as compared to a lower stability score that indicates a greater movement of mobile device D by the user. In one non-limiting example, the stability score can be calculated as a percentage of time that the mobile device D is held immobile by the user divided by the total length (duration) of the balance/stability exercise(s)/pose(s) performed by the user. In one embodiment, the stability algorithm module implemented by the device D and other aspects of the present system can be configured such that small movements of the device D below a certain threshold movement is considered to indicate that the device is “immobile” to compensate for minimal inherent movement of the device D by all users. Further, the velocity of movement in a unit of time exceeding specific pre-defined thresholds may help quantify the percent of time a user has poor (“meh”), good, or great stability. However, multiple metrics may be measured and combined as the scientific research surrounding stability measurement indicates no single perfect metric. Metrics described in the scientific literature for measurement of balance and stability include phase plane parameter, sway density, sway length, 95% confidence ellipse, fractal dimension, critical point coordinates from diffusion analysis, and more.

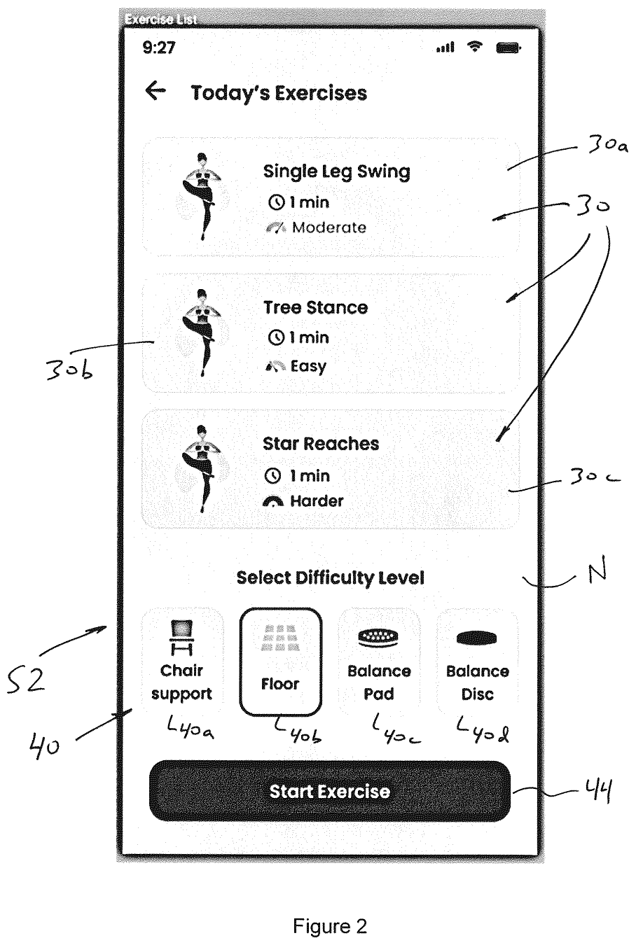

When the user selects the Start Today's Exercises icon 16 from the home screen S1 or otherwise begins the balance testing method, the mobile device display screen N can be updated to display the Today's Exercises screen S2, an example of which is shown in FIG. 2. The Today's Exercises screen S2 can include one or more selectable exercise icons 30 displayed to the user. In the illustrated non-limiting example, the selectable exercise icons 30a,30b,30c are respectively associated with a single leg swing exercise, a tree stance exercise, and a star reaches exercise, but other exercises or poses can be displayed and selected by the user. The Today's Exercises screen S2 can also include one or more Difficulty Level icons or elements 40 that can optionally be selected by the user to provide input to the device D as to the type of surface or support device on which the user is standing or if the user is sitting in or otherwise utilizing a chair or other auxiliary support device for support. As shown, the Difficulty Level icons 40a,40b,40c,40d can be selected by a user to input a chair support device, a floor surface, a balance pad device, and a balance disc device, respectively. However, additional balance tools may be implemented such as a wobble board. During the initial onboarding of a user, the user can be asked questions pertaining to factors which may affect balance such as age, take a quantitative measure of balance stability, and use this information to tailor the user interface to show or hide certain options for training. In one embodiment, this would specifically limit the presented training exercises and surfaces available. For example, an 80-year-old user might be shown chair support or floor as the only available surfaces for the Difficulty Level icons 40, while a 20-year-old athlete may be offered only floor, balance pad, balance disk, and wobble board surfaces for selectable Difficulty Level icons 40. In one example, the Difficulty Level icon 40b associated with a standard floor support surface is selected by default unless/until the user selects a different Difficulty Level icon 40.

With continuing reference to FIG. 2, when the user has selected one of the exercises 30 and has selected any applicable Difficulty Level icon 40, the user can select the Start Exercise icon 44 and/or can verbally instruct the device D to begin the balance testing process as shown in FIG. 3.

As noted above, and as shown in FIG. 3 and/or FIG. 3A, when the user selects the Start Exercise icon 44, an exercise or pose screen S3 is displayed to the user on the device screen N. The displayed exercise screen S3 prompts the user visually on the display screen of the device D and/or via audible messages or tones output by the speaker SK of the mobile device D to perform one or more various balance and/or mobility exercises and/or poses via videos and/or images displayed on the screen N, preferably demonstrated by an image and/or video of a human or animated instructor I, that the user will mimic to the best of the user's ability, such as the Flamingo Pose example shown in FIG. 3 or the Tree Stance example shown in FIG. 3A. A user stability module of the device D implements an algorithm within the processor PX, sensors MX, memory MM, and/or other components of the device D or an associated device that receives as input movement measurements from the device D (X, Y, and Z axis movements and/or acceleration data from the device movement sensors MX) and that calculates or otherwise derives a “stability score” associated with the particular user that is indicative or representative of the user's stability while performing the one or more balance/mobility exercises or poses as instructed by the mobile device D by the exercise screen S3 displayed to the user. A higher stability score indicates a higher percentage of time that the mobile device D was held stable by the user while performing each balance/mobility exercise or pose as compared to a lower stability score that indicates a lower percentage of time that the mobile device D was held stable by the user while performing each balance/mobility exercise or pose. As shown in FIGS. 3 and 3A, the stability score SC can be graphically (FIGS. 3 and 3A) and/or alphanumerically displayed (FIG. 3A) to the user by a stability score element SC of the screen S3, along with a Time element T displayed to the user as part of the exercise screen S3 that graphically and/or alphanumerically counts/moves up or down to provide information to the user as to the time remaining for the user to perform the displayed exercise/pose (or the Time element T can alternatively count/move up or down or otherwise graphically and/or alphanumerically indicate time remaining for the exercise or workout). The device D can also display a difficulty icon or symbol or alphanumeric message DS that indicates the difficulty level selected by the user using the Difficulty Level icons 40 (shown in FIG. 3 as a Balance Disc corresponding to user selection of the Balance Disc icon 40d).

The user is successively presented with one or more exercise screens S3 and performs one or more exercises/poses according to the example of FIG. 3, but with different exercises and/or poses being displayed to the user for each successive exercise screen S3.

In one alternative embodiment, the device D includes a selfie camera CAM and the device captures video and/or still images of the user while the user is performing the balance exercise or pose. The captured image and/or video data can be displayed to the user on the device screen N together with the exercise/pose screen S3, e.g., as a smaller inset image/video or a side-by-side image layout. Also, the memory MM can store the captured selfie image/video data and the processor PX or another processor can perform artificial intelligence (AI) and other analysis on the image/video to assess the user's technique, to verify the user's compliance, and/or to assess the user's stability, balance, and/or flexibility.

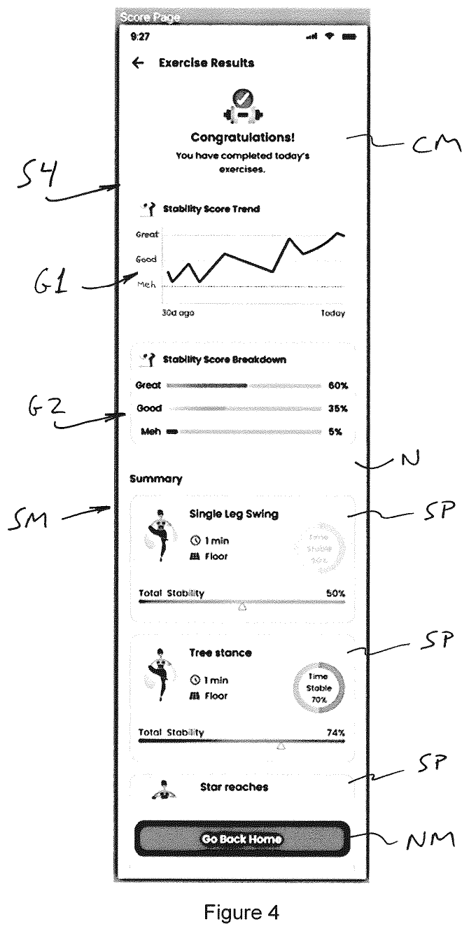

When the user has completed all exercises/poses, the user can be presented with an Exercise Results screen on the device display N, an example of which is shown in FIG. 4 at S4. This Exercise Results screen S4 can include the information shown in FIG. 4 or more or less information such as a graph G1 that graphically shows the user's stability score trend. A graphical analysis G2 of the user's stability scores, i.e., the percentage of the user's stability scores that fall within particular ranges such as the illustrated “Great” “Good” and “Meh” (poor) can also be displayed on the Exercise Results screen S4. As shown in FIG. 4, the Exercise Results screen S4 can also include a summary SM that includes one or more summary panels SP respectively associated with each of the exercises/poses performed by the user in an exercise session. In one example, as shown herein, each summary panel SP can display the total length (duration) of the exercise/pose, the associated selected difficulty level, a summary of the duration of time the device D was considered to be stable during the exercise/pose, and an overall stability score for that exercise. The user can scroll through each exercise/pose Summary Panel SP to see such information for each performed exercise or pose. The Exercise Results screen can also include a “Go Back Home” button or navigation menu NM that returns the device display N to the home screen S1 when selected by the user. The Exercise Results screen S4 can also include a congratulatory message CM that congratulates the user on completing an exercise/pose routine. Any or all of the information set forth on the Exercise Results screen S4 can additionally and/or alternatively be output using audible output via device speaker SK.

FIG. 5 shows an example of a My Progress screen S5 that can be displayed to the user on the display N of the device D to provide the user with collection of visual information/data relating to the progress of the user with respect to the user's balance/stability as measured by the device D during the exercises/poses performed by the user. The progress data of the My Progress screen S5 can include a graph G5 that provides a graphical representation of the user's stability score trend over a weekly, monthly, quarterly, yearly, or other time period. The progress data of the My Progress screen S5 can also include a graphical representation G6 of the user's activity using the device D to perform exercises/poses, i.e., how often the user is using the device to perform stability exercises and poses as described herein, and can also display an Activity Summary AS element that provides graphical and/or alphanumeric information concerning the number of active days, consecutive days, active weeks, and/or consecutive weeks or other time periods where the user used the device to perform one or more stability exercises/poses. As shown at CMP, the My Progress screen S5 can also include a graphical and/or alphanumeric display element that provides an indication to the user of the user's stability score in comparison to other users such as other users of a similar age. Any or all of the information set forth on the My Progress screen S5 can additionally and/or alternatively be output using audible output via device speaker SK.

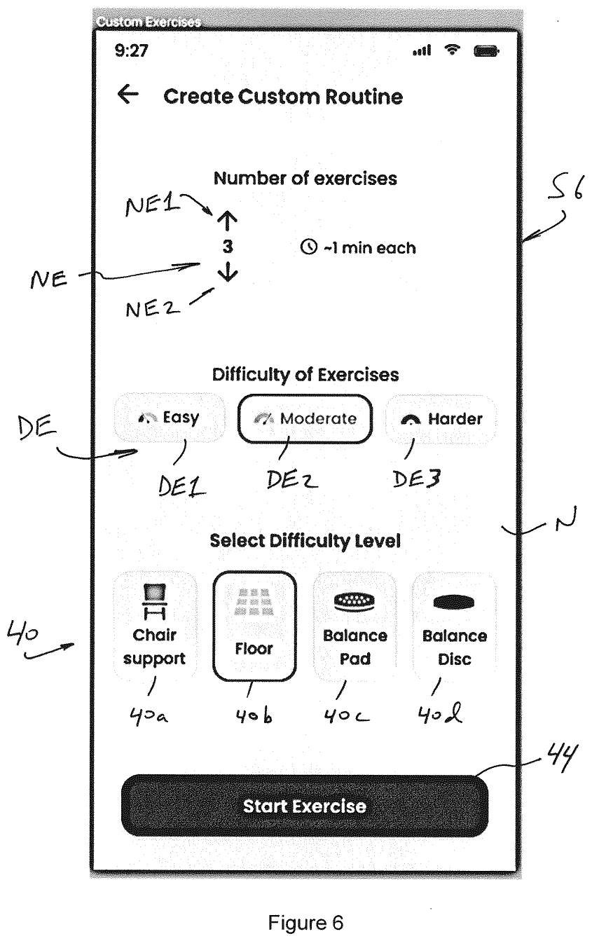

When the user selects the Create Custom Routine icon 20 from the home screen S1, the user can be presented with a Create Custom Routine screen S6 on the device display N as shown in FIG. 6. The Create Custom Routine screen S6 can include a Number of Exercises selector element NE that allows the user to increase or decrease the number of exercises or poses to be performed in each session by selecting the up or down buttons NE1,NE2 on the display N of the device D. The Create Custom Routine screen S6 can include a Difficulty of Exercises selector element DE that allows the user to increase or decrease the difficulty of the exercises or poses to be performed in each exercise session such as, for example, selecting the easy, moderate or harder buttons DE1,DE2,DE3 on the display N of the device D. The Create Custom Routine screen S6 can include a Difficulty Level selector element 40 (as described above in relation to the Today's Exercises screen S2) that allows the user to select the difficulty of the support surface on which the user is supported such as, for example, by allowing the user to select the chair support, floor, balance pad, or balance disc buttons 40a,40b,40c,40d on the display N of the device D.

FIG. 7 shows a representative screenshot of a settings screen S7 that can be presented to the user on the display N of the device. The settings screen S7 displays various settings, biographical information, ability to refer a friend, leave a review, or contact the app provider/administrator that can be edited or otherwise manipulated by the user of the device D to configure various aspects of the system and device D.

In one embodiment, to ensure uniformity across exercises/poses and to help users understand stability, training data would be used to normalize stability scores across exercises, so that the stability score from one exercise may be compared with another, and so users can trend progress of stability over time regardless of whether performing the exact same exercise and/or the exact same duration. For example, a scalar multiplication of balance metrics or the final stability score may help normalize the differing difficulty, such as between standing tandem stance and standing on one leg. These scalars may be obtained by regression analysis of collected data from a group of training users. The application provides users with feedback on performance and may provide advice for means of improvement.

Not only should differing exercises have a modifiable difficulty, but also different surfaces or supports. Using a chair as support is one means of making many balance exercises safer for those with underlying instability while using devices including but not limited to a balance pad, balance disc, or wobble board create escalating levels of difficulty over a user standing on a solid floor surface. Normalizing the stability score based on the surface or support used allows the user to trend stability scores over time regardless of the surface used previously.

In addition, the stability score is preferably normalized to account for sway or tremors of the arm. This is performed, for example, by having the user assume a particular position of stability, which in one embodiment is standing with both feet planted, and positioning the device similarly as described above for use with exercises, and measuring data while the user maintains this position of stability for a pre-defined amount of time. This pre-defined amount of time in one embodiment is twenty seconds, although other time amounts may be used without departing from the scope and intent of the present disclosure. Depending on the calculation method used, subtraction, division, z score analysis, or other means of normalization could be performed, though variance in each measured dimension is convenient as this can be simply subtracted from the variance of each measured dimension after an exercise is completed. Users may be encouraged to take multiple baseline measurements, so as to have a more accurate representation of the user's arm tremors, as this has a significant impact on the overall stability score. The average, median, or some percentile can be used for each dimension of baseline stability.

Previous research has identified that people with poor balance have significantly higher phase plane parameters derived from measurements of Center of Pressure (COP) on a force platform. The phase plane parameter is the square root of sum of the variation of position change and variation in the velocity. Instead of COP on a force platform, using the device's D position in space and velocity data in 3-dimensional space and calculating a 3-dimensional phase plane parameter is a means of calculating stability is advantageous in its low computational complexity. In using a 3-dimensional phase plane parameter as a metric for calculation of stability, the stability score would be adjusted by subtracting the baseline phase parameter calculated while a user held a device D still while standing in a steady planted stance from the phase parameter calculated from the user's performance in an exercise with device D.

In other words, the device D can be used to measure the stability of a user by having the user hold the device with an outstretched arm or in a similar position and measuring the stability of the user while the user simply stands on two feet without performing a particular pose or exercise, to provide baseline stability data that indicates a user's general balance or stability.

While the above description enables static balance exercises to be performed and measured, dynamic balance exercises are also a desired, and typically necessary, component of balance training. To enable such functionality, when performing a specific dynamic exercise, a simple embodiment is that the algorithm will ignore, diminish the effect of, or augment one or two pre-determined axis or axes of movement of the accelerometer and/or specific angular movements of the gyroscope. In one embodiment, this would be performed by scalar multiplication of the x, y, and z accelerometer and x, y, and z gyroscope data. Multiplying each parameter by 1 might be the default for a static exercise such as tree stance. In contrast, a dynamic exercise such as a squat might apply a scalar multiple of 0 to movement in the y axis of accelerometer data, so as to ignore the up/down movements of the user. To ensure conformity of stability scores between different exercises, the x and z axes may then be augmented with a scalar multiple of 1.5. Certain exercises may ignore or diminish the effects of multiple axes, such as with a lunge, wherein user movements in both y and z axes are intentional. This scalar multiplication may be beneficial even with static exercises however, such as with the tandem stance, in which research has shown that left-right movement as measured by the x axis of the accelerometer is more predictive of poor balance than forward-back movement as measured by the z axis of the accelerometer. As such, a tactic of assigning a scalar multiple between 0-1 for the z axis and greater than 1 for the x axis may lead to more success in the prediction of a user's balance. For all exercises, the y axis may be diminished compared to x and z axes, as y axis movement is more likely secondary to inadvertent user arm movements than poor balance. The stability scores would be normalized, so as described above, a dynamic exercise stability score could be compared to a static exercise stability score, by utilizing training data. As discussed above, the application of a data filter such as a Butterworth fourth order filter with a predefined cutoff of 2-4 Hz or other frequency analysis filtering technique may augment or alternatively be used to ignore intentional user movement, as unintentional movements are more likely to have higher frequency components. An additional yet more computationally complex embodiment may include wavelet transforms or empirical mode decomposition methodologies which would detect and separate repetitive low frequency intentional movements of a user from higher-frequency inadvertent movements.

An additional performance metric may be tracked by the software by measuring the total distance the user moves the device for certain dynamic exercises, with the goal of the user improving their balance to the point where they can increase their total movement during an exercise and receive feedback on performance. For example, a functional reach test involves a person standing with both feet planted, arms outstretched, and the person reaches as far forward as possible. Greater reach is predictive of improved balance and may be tracked by the software as a metric. In addition, the algorithm may measure repeated movements over time such as going from the initial position to the final position of the functional reach test and calculate a score based on average distance moved. Additional measured variables that may factor into this score include smoothness of the user's movement and consistency of distance travelled. These performance metrics may be utilized as inputs affecting overall stability score or be listed as individual variables shown to the user to demonstrate different aspects of stability.

As a safety measure, users with low stability scores may receive a recommendation from the application to reduce the difficulty level of the exercises, to reduce fall risk. Users who are already at the lowest level of difficulty of exercises performed by the app in the manner described above may then be prompted to perform even simpler exercises for which performance tracking may not be implemented. Examples include seated or floor exercises such as ankle circles, seated heel taps, and boat pose. Additionally, the algorithm may be able to determine a user is cheating by setting the device on a stable surface such as a table if the device does not move beyond a threshold minimum total distance.

For additional safety, the algorithm may detect jerking movements and falls using thresholds of acceleration for both data from the accelerometer and gyroscope, and penalize the user or even lock the user from using the app. For example, in one embodiment, users may receive points every interval of time such as 10 seconds, and users lose points for each jerk detected, whereas a fall would immediately end the exercise and cause the loser to lose all points. Additionally, the app may downgrade the user's difficulty level of balance training exercises or lock the user from using the app if exceeding a certain number of total falls or falls within a specific timeframe or number of completed exercises.

While the subject matter of the present disclosure has been described with reference to the foregoing embodiments and considerable emphasis has been placed herein on the structures and structural interrelationships between the component parts or steps of the embodiments disclosed, it will be appreciated that other embodiments can be made or implemented and that many changes can be made in the embodiments illustrated and described without departing from the principles hereof. Obviously, modifications and alterations will occur to others upon reading and understanding the preceding detailed description. Accordingly, it is to be understood that the foregoing descriptive matter is to be interpreted merely as illustrative of the subject matter of the present disclosure and not as a limitation. As such, it is intended that the subject matter of the present disclosure be construed as including all such modifications and alterations while maintaining the validity of the following claims.

I claim:

1. A system for testing or improving balance of an associated user, the system comprising:
a wearable or holdable device;
at least one sensor that senses movement of the device and transmits movement data to a processor operatively associated therewith; and
said processor configured to use the movement data from the at least one sensor to provide feedback representing balance or stability of the associated user.

2. The system of claim 1, wherein the device includes the sensor and processor and a memory.

3. The system of claim 2, wherein the device includes a display that shows the associated user a lesson or exercise to perform.

4. The system of claim 3, wherein the at least one sensor includes an accelerometer and/or a gyroscope.

5. The system of claim 4, wherein the lesson or exercise pertaining to balance or mobility is shown on the display to the associated user.

6. The system of claim 5, wherein movement of the device is measured by the accelerometer and/or gyroscope of the device while the associated user performs the lesson or exercise.

7. A method for testing or improving balance of an associated user using a wearable or holdable device, the method comprising:
wearing or holding the device;
sensing movement of the device with at least one sensor and transmitting movement data to a processor operatively associated therewith; and
using the movement data from the at least one sensor to provide feedback representing balance of the associated user.

8. The method of claim 7, further comprising including the at least one sensor and processor in the device.

9. The method of claim 8, further comprising showing the associated user a lesson or exercise to perform on a display associated with the device.

10. The method of claim 9, further comprising providing an accelerometer and/or a gyroscope as part of the at least one sensor.

11. The method of claim 10, further comprising showing the lesson or exercise pertaining to balance or mobility on the display to the associated user.

12. The method of claim 11, further comprising measuring movement of the device with the accelerometer and/or gyroscope of the device while the associated user performs the lesson or exercise.

13. The method of claim 12, further comprising using an algorithm to convert the measured movements to a stability score.

14. The method of claim 13, further comprising augmenting, diminishing, or ignoring the measured movements in multiple dimensions for specific exercises.

15. The method of claim 13, further comprising providing real-time feedback to the associated user on current stability.

16. The method of claim 15, wherein providing the real-time feedback includes at least one of a one-dimensional graph or multi-dimensional vector representation.

17. The method of claim 13, further comprising adjusting the score to account for arm sway and tremors by use of a baseline measurement.

18. The method of claim 13, further comprising using training data to normalize scores across exercises and different surfaces on which the user performs the exercise so that a stability score can be compared across exercises and regardless of duration of the exercise.

19. The method of claim 13, further comprising measuring movements to calculate distance traveled and smoothness of the movement with each repetition of an exercise, then calculating performance metric(s) based on the average or median distance per repetition, the standard deviation of the distances with each repetition, and the average or deviations of smoothness of the movement.

20. The method of claim 19, further comprising using the performance metrics (i) to be shown to the associated user and/or (ii) as inputs into calculating a stability score.

21. The method of claim 20, further comprising normalizing the stability score against arm movements by taking a pre-training assessment.

22. The method of claim 21, further comprising determining a final stability score at the end of a given exercise or collection of exercises.

23. The method of claim 22, further comprising normalizing the final additive stability score for a duration of the exercise performed.

24. The method of claim 19, further comprising providing trends to the associated user regarding the stability score and/or performance metrics.

25. The method of claim 19, further comprising advising the associated user to perform less risky exercises to mitigate fall risk in response to low stability scores or falls.
